(19) United States
(12) Patent Application Publication
(10) Pub. No.: US 2003/0040506 A1
(43) Pub. Date: Feb. 27, 2003

(54) Analogs of (1s,cis)-4-(2-amino-9h-purin -9-yl) -2-Cyclopentene-1-methanol as antiviral

(76) Inventor: Susan Mary Daluge, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/149,198

(22) PCT Filed: Dec. 7, 2000
(86) PCT No.: PCT/US00/33204

(51) Int. Cl. A61K 31/675; C07F 9/6512

(52) U.S. Cl. 514/81; 544/244

(57) ABSTRACT

The present invention relates to analogs of (1S, cis)-4-(2-amino-9H-purin-9-yl)-2-cyclopentene-1-methanol for use in treating viral infections.

### FIELD OF THE INVENTION

The present invention relates to phosphoramidates of 6-modified analogs of (1S, cis)-4-(2-amino-9H-purin-9-yl)-2-cyclopentene-1-methanol and their use in medical therapy.

### BACKGROUND OF THE INVENTION

Retroviruses form a sub-group of RNA viruses which, in order to replicate, must first “reverse transcribe” the RNA of their genome into DNA (“transcription” conventionally describes the synthesis of RNA from DNA) Once in the form of DNA, the viral genome may be incorporated into the host cell genome, allowing it to take advantage of the host cell's transcription/translation machinery for the purposes of replication. Once incorporated, the viral DNA is virtually indistinguishable from the host's DNA and, in this state, the virus may persist for the life of the cell.

A species of retrovirus, the Human immunodeficiency virus (HIV) has been reproducibly isolated from patients with AIDS (acquired immunodeficiency syndrome) or with the symptoms that frequently precede AIDS. AIDS is an immunosuppressive or immunodestructive disease that predisposes subjects to fatal opportunistic infections. Characteristically, AIDS is associated with a progressive depletion of T-cells, especially the helper-inducer subset bearing the CD4 surface marker. HIV is cytopathic and appears to preferentially infect and destroy T-cells bearing the CD4 marker, and it is now generally recognized that HIV is the etiological agent of AIDS. Clinical conditions such as AIDS-related complex (ARC), progressive generalized lymphadenopathy (PGL), Karposi's sarcoma, thrombocytopenic purpura, AIDS-related neurological conditions, such as AIDS dementia complex, multiple sclerosis or tropical paraparesis, and also anti-HIV antibody-positive and HIV-positive conditions, including such conditions in asymptomatic patients, are also conditions which may be treated by appropriate anti-viral therapy.

Another RNA virus which has been recognized as the causative agent of an increasingly serious international health problem is the non-A, non-B hepatitis virus. At least 80% of cases of chronic post-transfusional non-A, non-B hepatitis have been shown to be due to the virus now identified as hepatitis C and this virus probably accounts for virtually all cases of post-transfusional hepatitis in clinical settings where blood products are screened for hepatitis B. Whereas approximately half of the cases of acute hepatitis C infection resolve spontaneously over a period of months, the remainder become chronic and in many if not all such cases chronic active hepatitis ensues with the potential for cirrhosis and hepatocellular carcinoma. The structure of the hepatitis C virus genome has been elucidated and the virus has been characterized as a single stranded RNA virus with similarities to flaviviruses.

Hepatitis B virus (HBV) is a small DNA containing virus which infects humans. It is a member of the class of closely related viruses known as the hepadnaviruses, each member of which selectively infects either mammalian or avian hosts, such as the woodchuck and the duck. Recent insights into the mechanism of replication of the hepadnavirus genome indicate the importance of reverse transcription of an RNA intermediate, suggesting that the reverse transcriptase is a logical chemotherapeutic target. HBV is a viral pathogen of major world-wide importance. The virus is etiologically associated with primary hepatocellular carcinoma and is thought to cause 80% of the world's liver cancer. Clinical effects of infection with HBV range from headache, fever, malaise, nausea, vomiting, anorexia and abdominal pains. Replication of the virus is usually controlled by the immune response, with a course of recovery lasting weeks or months in humans, but infection may be more severe leading to persistent chronic liver disease outlined above.

U.S. Pat. No. 4,916,224 discloses 2′,3′-dideoxy-2′,3′-didehydro-carbocyclic nucleosides and their use in the treatment of HIV. U.S. Pat. No. 5,049,671 discloses 6-substituted purine carbocyclic nucleosides and their use in medical therapy particularly in the treatment of HIV and HBV infections. European patent No. EP 0349242A2 discloses 6-substituted purine carbocyclic nucleosides and their use in medical therapy, particularly in the treatment and prophylaxis of HIV and HBV infections. WO 96/29336 discloses masked monophosphate nucleoside analogues for the treament of HIV.

It has now been discovered that certain phosphoramidates of 0.6-modified analogs of (1S, cis)-4-(2-amino-9H-purin-9-yl)-2-cyclopentene-1-methanol are useful for the treatment of viral infections, particularly hepatitis B and hepatitis C and retroviral infections, especially HIV. Compounds of the present invention have pharmacokinetic properties which render them advantageous as therapeutic agents.

### SUMMARY OF THE INVENTION

The present invention relates to compounds of formula (I)

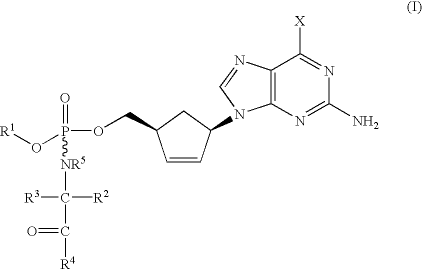

R¹ is hydrogen; C₆₋₁₄aryl; or heteroaryl, optionally substituted with one or more substituents selected from the group consisting of C₁₋₆alkoxy, nitro, halogen, amino, hydroxy, carboxylate and esters thereof, carboxyalkyl, —CONHR⁶, and —CONR⁶R⁷, wherein R⁶ and R⁷, which may be the same or different, are independently selected from C₁₋₈alkyl, C₁₋₈alkylarvl or C₆₋₁₄aryl;

R² and R³ are independently selected from hydrogen or C₁₋₈alkyl, C₃₋₈cycloalkyl, C₂₋₈alkenyl, C₅₋₈cycloalkenyl, C₆₋₁₄aryl, aralkyl wherein each C₁₋₈alkyl, C₃₋₈cycloalkyl, C₂₋₈alkenyl, C₅₋₈cycloalkenyl, C₆₋₁₄aryl or aralkyl may be optionally substituted with one or more substituents selected from the group consisting of C₁₋₈alkyl, halo, hydroxy, alkoxy, amino, aminoalkyl, aminodialkyl, —SH, thioalkyl, carboxylate and esters thereof, carboxyalkyl, —CONHR⁶, and —CONR⁶R⁷, wherein R⁶ and R⁷, which may be the same or different, are independently selected from C₁₋₈alkyl, C₁₋₈alkylaryl or C₆₋₁₄aryl; or R² and R³ can together form a 3- to 8-membered ring;

R⁴ is —OR⁸, —NR⁸R⁹ or —SR⁸, where R⁸ and R⁹, which may be the same or different, are independently selected from hydrogen, or C₁₋₈alkyl, C₃₋₈cycloalkyl, C₂₋₈alkenyl, C₅₋₈cycloalkenyl, or aralkyl, wherein each C₁₋₈alkyl, C₃₋₈cycloalkyl, C₂₋₈alkenyl, C₅₋₈cycloalkenyl, or aralkyl may be optionally substituted with one or more substituents selected from the group consisting of halo, hydroxy, alkoxy, amino, aminoalkyl, aminodialkyl, —SH, thioalkyl, carboxylate and esters thereof, carboxyalkyl, —CONHR⁶, and —CONR⁶R⁷, wherein R⁶ and R⁷, which may be the same or different, are independently selected from C₁₋₈alkyl, C₁₋₈alkylaryl or C₆₋₁₄aryl;

R⁵ is hydrogen; C₁₋₈alkyl; or C₆₋₁₄aryl; or R² and R⁵ may together form a 5- or 6-membered ring; or R³ and R⁵ may together form a 5- or 6-membered ring;

X is C₁₋₆alkoxy, optionally substituted by C₃₋₆cycloaklyl; C₃₋₈cycloalkyloxy; aryloxy, aralkyl or aralkyloxy in which the aryl may optionally be substituted with C₁₋₈alkyl, hydroxy, or halogen; C₃₋₆cycloalkylthio; C₁₋₈alkylthio; arylthio, or aralkylthio in which the aryl may optionally be substituted with C₁₋₈alkyl, hydroxy, or halogen; or X is a heterocyclic group containing an oxygen atom or one or two nitrogen atoms, and 3-7 carbon atoms with optional double bonds in the ring, optionally containing a sulfur and/or oxygen heteroatom and optionally substituted on the ring by one or more C₁₋₈alkyl, hydroxy or halogen groups, C₃₋₆cycloalkylthio, aralkylthio in which the aryl may be substituted with C₁₋₈alkyl, hydroxy or halogen; or X represents an imidazolylthio group in which the imidazolyl moiety may be substituted with C₁₋₈alkyl and/or C substituted with nitro; or X represents an amino group which is mono- or di-substituted by C₁₋₈alkyl, C₁₋₆alkoxy, hydroxyC₁₋₈alkyl and/or C₃₋₆cycloalkyl, C₆₋₁₄aryl, aralkyl, in which the C₆₋₁₄aryl may be optionally substituted with C₁₋₈alkyl, hydroxy, halogen or allyl optionally substituted with mono- or di-alkyl or alkoxy groups;

or a pharmaceutically acceptable derivative thereof; provided that X cannot be amino monosubstituted with cyclopropyl. The present invention relates to the use of compounds of formula (I) in the treatment of viral infections.

### DETAILED DESCRIPTION OF THE INVENTION

The present invention features compounds of formula (I)

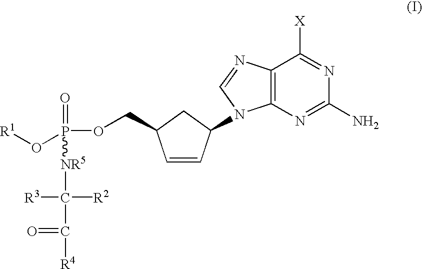

wherein:

R¹ is hydrogen; C₆₋₁₄aryl; or heteroaryl, optionally substituted with one or more substituents selected from the group consisting of C₁₋₆alkoxy, nitro, halogen, amino, hydroxy, carboxylate and esters thereof, carboxyalkyl, —CONHR⁶, and —CONR⁶R⁷, wherein R⁶ and R⁷, which may be the same or different, are independently selected from C₁₋₈alkyl, C₁₋₈alkylaryl or C₆₋₁₄aryl;

R² and R³ are independently selected from hydrogen or C₁₋₈alkyl, C₃₋₈cycloalkyl, C₂₋₈alkenyl, C₅₋₈cycloalkenyl, C₆₋₁₄aryl, aralkyl wherein each C₁₋₈alkyl, C₃₋₈cycloalkyl, C₂₋₈alkenyl, C₅₋₈cycloalkenyl, C₆₋₁₄aryl or aralkyl may be optionally substituted with one or more substituents selected from the group consisting of C₁₋₈alkyl, halo, hydroxy, alkoxy, amino, aminoalkyl, aminodialkyl, —SH, thioalkyl, carboxylate and esters thereof, carboxyalkyl, —CONHR⁶, and —CONR⁶R⁷, wherein R⁶ and R⁷, which may be the same or different, are independently selected from C₁₋₈alkyl, C₁₋₈alkylaryl or C₆₋₁₄aryl; or R² and R³ can together form a 3- to 8-membered ring;

R⁴ is —OR⁸, —NR⁸R⁹ or —SR⁸, where R⁸and R⁹, which may be the same or different, are independently selected from hydrogen, or C₁₋₈alkyl, C₃₋₈cycloalkyl, C₂₋₈alkenyl, C₅₋₈cycloalkenyl, or aralkyl, wherein each C₁₋₈alkyl, C₃₋₈cycloalkyl, C₂₋₈alkenyl, C₅₋₈cycloalkenyl, or aralkyl may be optionally substituted with one or more substituents selected from the group consisting of halo, hydroxy, alkoxy, amino, aminoalkyl, aminodialkyl, —SH, thioalkyl, carboxylate and esters thereof, carboxyalkyl, —CONHR⁶, and —CONR⁶R⁷, wherein R⁶ and R⁷, which may be the same or different, are independently selected from C₁₋₈alkyl, C₁₋₈alkylaryl or C₆₋₁₄aryl;

R⁵ is hydrogen; C₁₋₈alkyl; or C₆₋₁₄aryl; or R² and R⁵ may together form a 5- or 6-membered ring; or R³ and R⁵ may together form a 5- or 6-membered ring;

X is C₁₋₆alkoxy, optionally substituted by C₃₋₆cycloaklyl; C₃₋₈cycloalkyloxy; aryloxy, aralkyl or aralkyloxy in which the aryl may optionally be substituted with C₁₋₈alkyl, hydroxy, or halogen; C₃₋₆cycloalkylthio; C₁₋₈alkylthio; arylthio, or aralkylthio in which the aryl may optionally be substituted with C₁₋₈alkyl, hydroxy, or halogen; or X is a heterocyclic group containing an oxygen atom or one or two nitrogen atoms, and 3-7 carbon atoms with optional double bonds in the ring, optionally containing a sulfur and/or oxygen hetenoatom and optionally substituted on the ring by one or more C₁₋₈alkyl, hydroxy or halogen groups, C₃₋₆cycloalkylthio, aralkylthio in which the aryl may be substituted with C₁₋₈alkyl, hydroxy or halogen; or X represents an imidazolylthio group in which the imidazolyl moiety may be substituted with C₁₋₈alkyl and/or C substituted with nitro; or X represents an amino group which is mono- or di-substituted by C₁₋₈alkyl, C₁₋₆alkoxy, hydroxyC₁₋₈alkyl and/or C₃₋₆cycloalkyl, C₆₋₁₄aryl, aralkyl, in which the C₆₋₁₄aryl may be optionally substituted with C₁₋₈alkyl, hydroxy, halogen or allyl optionally substituted with mono- or di-alkyl or alkoxy groups;

or a pharmaceutically acceptable derivative thereof; provided that X cannot be amino monosubstituted with cyclopropyl.

The compounds of the present invention include diastereomers differing in the absolute configuration at phosphorus. Diastereomers may be present as a single isomer or as mixtures of diastereomers.

Where R² and R³ are different, the L-configuration of naturally occurring amino acids is preferred.

The term “alkyl” refers to a straight-chain or branched-chain saturated aliphatic hydrocarbon radical containing the specified number of carbon atoms, or where no number is specified, preferably from 1 to about 10, more preferably from 1 to about 8 carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like.

The term “aryl” refers to a carbocyclic aromatic radical (such as phenyl or naphthyl) containing the specified number of carbon atoms, preferably from 6-14 carbon atoms, and more preferably from 6-10 carbon atoms, optionally substituted with one or more substitutents selected from C₁₋₆ alkoxy (for example, methoxy), nitro, halogen (for example chloro), amino, carboxylate and hydroxy. Examples of aryl radicals include, but are not limited to phenyl, naphthyl, indenyl, indanyl, azulenyl, fluorenyl, anthracenyl and the like.

The term “alkenyl,” alone or in combination with any other term, refers to a straight-chain or branched-chain mono- or poly-unsaturated aliphatic hydrocarbon radical containing the specified number of carbon atoms, or where no number is specified, preferably from 2-10 carbon atoms and more preferably, from 2-6 carbon atoms. Examples of alkenyl radicals include, but are not limited to, ethenyl, propenyl, isopropenyl, butenyl, isobutyenyl, pentenyl, hexenyl, hexadienyl and the like.

The term “alkoxy” refers to an alkyl ether radical, wherein the term “alkyl” is defined above. Examples of suitable alkyl ether radicals include, but are not limited to, methoxy, ethoxy, n-propoxy, isopropoxy, n-butoxy, isobutoxy, sec-butoxy, tert-butoxy and the like, with methoxy being preferred.

The term “halo” or “halogen” refers to a radical of fluorine, chlorine, bromine or iodine.

The term “heterocycle”, alone or in combination with another term, refers to a stable 3-7 membered monocyclic heterocyclic ring or 8-11 membered bicyclic heterocyclic ring which is either saturated or unsaturated, and which may be optionally benzofused if monocyclic. Each heterocycle consists of one or more carbon atoms and from one to four heteroatoms selected from the group consisting of nitrogen, oxygen and sulfur. As used herein, the terms “nitrogen and sulfur heteroatoms” include any oxidized form of nitrogen and sulfur, and the quaternized form of any basic nitrogen. A heterocyclyl radical may be attached at any endocyclic carbon or heteroatom which results in the creation of a stable structure. Preferred heterocycles include 5-7 membered monocyclic heterocycles and 8-10 membered bicyclic heterocycles. Examples of such groups include imidazolyl, imidazolinoyl, imidazolidinyl, quinolyl, isoqinolyl, indolyl, indazolyl, indazolinolyl, perhydropyridazyl, pyridazyl, pyridyl, pyrrolyl, pyrrolinyl, pyrrolidinyl, pyrazolyl, pyrazinyl, quinoxolyl, piperidinyl, pyranyl, pyrazolinyl, piperazinyl, pyrimidinyl, pyridazinyl, morpholinyl, thiamorpholinyl, furyl, thienyl, triazolyl, thiazolyl, carbolinyl, tetrazolyl, thiazolidinyl, benzofuranoyl, thiamorpholinyl sulfone, oxazolyl, benzoxazolyl, oxopiperidinyl, oxopyrrolidinyl, oxoazepinyl, azepinyl, isoxozolyl, isothiazolyl, furazanyl, tetrahydropyranyl, tetrahydrofuranyl, thiazolyl, thiadiazoyl, dioxolyl, dioxinyl, oxathiolyl, benzodioxolyl, dithiolyl, thiophenyl, tetrahydrothiophenyl, sulfolanyl, dioxanyl, dioxolanyl, tetahydrofurodihydrofuranyl, tetrahydropyranodihydrofuranyl, dihydropyranyl, tetradyrofurofuranyl and tetra hydropyranofuranyl.

The term “pharmaceutically acceptable derivative”, as used herein, means any pharmaceutically acceptable salt, ester, salt of an ester, or other derivative of a compound of this invention which, upon administration to a recipient, is capable of providing (directly or indirectly) a compound of this invention or an inhibitorily active metabolite or residue thereof. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species.

Compounds of formula (I) and their pharmaceutically acceptable derivatives may hereinafter be referred to as compounds according to the invention.

A further aspect of the present invention features a compound of formula (I) wherein R¹ is C₆₋₁₄aryl; R² and R³ are independently selected from hydrogen, C₁₋₈alkyl, C₆₋₁₄aryl or aralkyl optionally substituted with C₁₋₈alkyl; R⁴ is —OR⁸, where R⁸ is selected from C₁₋₈alkyl or aralkyl; R⁵ is hydrogen; and X represents C₁₋₆alkoxy or C₁₋₆alkylthio; or X represents a heterocyclic group containing a nitrogen atom, and 3-7 carbon atoms; or X represents an amino group which is mono- or di-substituted by C₁₋₆alkyl, C₁₋₆alkoxy, hydroxyC₁₋₆alkyl and/or C₃₋₆cycloalkyl, C₆₋₁₄aryl, aralkyl, in which the aryl may be optionally substituted with C₁₋₈alkyl, hydroxy, halogen or allyl optionally substituted with mono- or di-alkyl or alkoxy groups; or a pharmaceuticallly acceptable derivative thereof; provided that X cannot be amino monosubstituted with cyclopropyl.

Another aspect of the present invention features compounds of formula (I) selected from the group consisting of

(1S,cis)-4-[2-Amino-6-(1-azetidinyl)-9H-purin-9-yl]-2-cyclopentene-1-methanol O-[phenyl(methoxy-L-alaninyl)]phosphoramidate;

(1S,cis)-4-[2-Amino-6-(cyclopropylmethylamino)-9H-purin-9-yl]-2-cyclopentene-1-methanol O-[phenyl(methoxy-L-alaninyl)]phosphoramidate;

(1S,cis)-4-(2-Amino-6-butoxy-9H-purin-9-yl)-2-cyclopentene-1-methanol O-[phenyl(methoxy-L-alaninyl)]phosphoramidate;

(1S,cis)-4-(2-Amino-6-methoxy-9H-purin-9-yl)-2-cyclopentene-1-methanol O-[phenyl(methoxy-L-alaninyl)] phosphoramidate;

(1S,cis)-4-[2-Amino-6-(propylamino)-9H-purin-9-yl]-2-cyclopentene-1-methanol O-[phenyl(methoxy-L-alaninyl)]phosphoramidate;

(1S,cis)-4-[2-Amino-6-(isopropylamino)-9H-purin-9-yl]-2-cyclopentene-1-methanol O-[phenyl(methoxy-L-alaninyl)]phosphoramidate;

(1S,cis)-4-[2-Amino-6-(allylthio)-9H-purin-9-yl]-2-cyclopentene-1-methanol O-[phenyl(methoxy-L-alaninyl)]phosphoramidate;

(1S,cis)-4-(2,6-Diamino-9H-purin-9-yl)-2-cyclopentene-1-methanol O-[phenyl(methoxy-L-alaninyl)]phosphoramidate;

(1S,cis)-4-[2-Amino-6-(cyclopropylmethylamino)-9H-purin-9-yl]-2-cyclopentene-1-methanol O-[phenyl(methoxy-α,α-dimethylglycinyl)]phosphoramidate;

(1S,cis)-4-[2-Amino-6-(cyclopropylmethylamino)-9H-purin-9-yl]-2-cyclopentene-1-methanol O-[phenyl(benzyloxy-L-alaninyl)]phosphoramidate;

(1S,cis)-4-[2-Amino-6-(cyclopropylmethylamino)-9H-purin-9-yl-2-cyclopentene-1-methanol O-[phenyl(methoxy-L-phenylalaninyl)]phosphoramidate;

and pharmaceutically acceptable derivatives thereof.

Physiologically acceptable salts of the compounds of the present invention include salts of a basic or acidic portion of the molecule. Salts of a basic moiety are formed by organic carboxylic acids such as acetic, lactic, tartaric, malic, isethionic, lactobionic, and succinic acids, organic sulphonic acids, such as miethanesulphonic, ethanesulphonic, benzenesulphonic and p-toluenesulphonic acids and inorganic acids, such as hydrochloric, sulphuric, phosphoric and sulphamic acids. Salts of an acidic moiety are formed by an appropriate base, such as an alkali metal (for example, sodium), an alkaline earth (for example, magnesium, calcium), ammonium and ammonium salts. For therapeutic use, salts of compounds according to the invention will be physiologically acceptable, i.e. they will be salts derived from a physiologically acceptable acid. However, salts of acids which are not physiologically acceptable may also find use, for example, in the preparation or purification of a physiologically acceptable compound. All salts, whether or not derived from a physiologically acceptable acid, are within the scope of the present invention.

Compounds of formula (I) may be made by modifications of the procedures described in Biochem. Biophys. Res. Commun. 225:363-369, 1996.

The present invention futher includes a process for the preparation of a compound according to the invention which comprises reaction of a compound of formula (II)

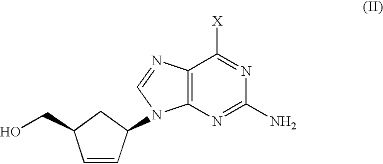

with a compound of formula (Ill)

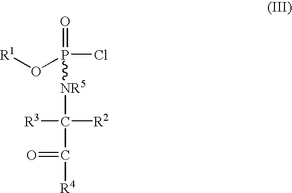

wherein R¹-R⁵ are as hereinbefore defined.

The reaction may be carried out in pyridine, pyridine-tetrahydrofuran, or acetonitrile in the presence of t-butyl magnesium chloride (Balzarini et al., Biochem. Biophys. Res. Comm. 225:363-369 (1996). The phosphochloridate intermediates may be prepared according to WO 96/29336.

Separation of isomers may be accomplished by methods known in the art, for example, by high-pressure liquid chromatography with chiral columns, particularly using liquid carbon dioxide as the mobile phase, or by crystallization of salts with chiral acids or bases.

Phosphate isomers may be separated with Supercritical Fluid Chromatography using a Chiralpak® AS column, 25% methanol in carbon dioxide as the eluent, flow rate 2 mL/min, temperature 40° C., and pressure 3000 psi.

One aspect of the invention features the compounds according to the invention for use in medical therapy, particularly for the treatment of retroviral infections, particularly HIV, and hepatitis B virus and hepatitis C virus infections.

A further aspect of the invention features the compounds according to the invention for use in the manufacture of a medicament for the treatment of viral infections, particularly for the treatment of retroviral infections, particularly HIV, and hepatitis B virus and hepatitis C virus infections.

In a further aspect of the present invention there is provided a method for the treatment of retroviral infections, and hepatitis B virus infections and hepatitis C virus infections in a host comprising administering to said host a therapeutically effective amount of a compound according to the invention.

Examples of retroviral infections which may be treated or prevented in accordance with the invention include human retroviral infections such as human immunodeficiency virus (HIV), HIV-1, HIV-2 and human T-cell lymphotropic virus (HTLV), for example, HTLV-I or HTLV-II infections. The compounds according to the invention are especially useful for the treatment of AIDS and related clinical conditions such as AIDS-related complex (ARC), progressive generalized lymphadenopathy (PGL), AIDS-related neurological conditions, such as multiple sclerosis or tropical paraparesis, anti-HIV antibody-positive and HIV-positive conditions and thrombocytopenic purpura.

The compounds according to the invention are particularly applicable for the treatment of asymptomatic infections or diseases in humans caused by or associated with human retroviruses.

The compounds according to the invention may be stable towards acid-mediated hydrolytic decomposition and thus, advantageous as therapeutic agents for oral administration, because the compounds are likely to withstand the acidic environment of the stomach.

The compounds according to the invention may be employed in combination with other therapeutic agents for the treatment of the above infections or conditions. Other therapeutic agents may include agents that are effective for the treatment of viral infections or associated conditions such as reverse transcriptase inhibitors, for example, zidovudine and abacavir; (1 alpha, 2 beta, 3 alpha)-9-[2,3-bis(hydroxymethyl)cyclobutyl]guanine [(−)BHCG, SQ-34514]; oxetanocin-G (3,4-bis-(hydroxymethyl)-2-oxetanosyl]guanine); acyclic nucleosides (e.g. acyclovir, valaciclovir, famciclovir, ganciclovir, penciclovir); acyclic nucleoside phosphonates (e.g. (S)-1-(3-hydroxy-2-phosphonyl-methoxypropyl)cytosine (HPMPC) or PMEA or PMPA; ribonucleotide reductase inhibitors such as hydroxyurea, 2-acetylpyridine 5-[(2-chloroanilino)thiocarbonyl) thiocarbonohydrazone; other 2′,3′-dideoxynucleosides such as 2′,3′-dideoxycytidine, 2′,3′-dideoxyadenosine, 2′,3′-dideoxyinosine, 3′-deoxy-2′,3′-didehydrothymidine (d4T); protease inhibitors such as saquinavir, indinavir, ritonavir, nelfinavir, amprenavir, tipranavir; oxathiolane nucleoside analogues such as lamivudine, cis-1-(2-(hydroxymethyl)-1,3-oxathiolan-5-yl)-5-fluorocytosine (FTC); 3′-deoxy-3′-fluorothymidine, 5-chloro-2′,3′-dideoxy-3′-fluorouridine, ribavirin, 9-[4-hydroxy-2-(hydroxymethyl)but-1-yl]-guanine (H2G); tat inhibitors such as 7-chloro-5-(2-pyrryl)-3H-1,4-benzodiazepin-2-(H)one (Ro5-3335), 7-chloro-1,3-dihydro-5-(1H-pyrrol-2yl)-3H-1,4-benzodiazepin-2-amine (Ro24-7429); interferons such as α-interferon; renal excretion inhibitors such as probenecid; nucleoside transport inhibitors such as dipyridamole; pentoxifylline, N-acetylcysteine (NAC), Procysteine, α-trichosanthin, phosphonoformic acid, as well as immunomodulators such as interleukin II or thymosin, granulocyte macrophage colony stimulating factors, erythropoetin, soluble CD4 and genetically engineered derivatives thereof; or non-nucleoside reverse transcriptase inhibitors (NNRTIs) such as nevirapine (BI-RG-587), loviride (α-APA), delavuridine (BHAP), atevirdine, efavirenz, and DPC 961/963, and phosphonoformic acid, and 1,4-dihydro-2H-3,1-benzoxazin-2-ones NNRTIs such as (−)-6-chloro-4cyclopropylethynyl-4-trifluoromethyl-1,4-dihydro-2H-3,1-benzoxazin-2-one (L-743,726 or DMP-266) and quinoxaline NNRTIs such as isopropyl (2S)-7-fluoro-3,4-dihydro-2-ethyl-3-oxo-1 (2H)-quinoxalinecarboxylate (HBY1293). The component compounds of such combination therapy may be administered simultaneously, in either separate or combined formulations, or at different times, for example, sequentially such that a combined effect is achieved.

The compounds according to the invention, also referred to herein as the active ingredient, may be administered for therapy by any suitable route including oral, rectal, nasal, topical (including buccal and sublingual), vaginal and parenteral (including subcutaneous, intramuscular, intravenous and intradermal). It will be appreciated that the preferred route will vary with the condition and age of the recipient, the nature of the infection and the chosen active ingredient.

The amounts required of the active ingredient will depend upon a number of factors including the severity of the condition to be treated and the identity of the recipient and will ultimately be at the discretion of the attendant physician or veterinarian. In general however, for each of these utilities and indications, a suitable effective dose of a compound of formula (I) will be in the range of 0.01 to 100 mg per kilogram body weight of recipient per day, advantageously in the range of 1 to 70 mg per kilogram body weight per day, preferably in the range of 1 to 50 mg per kilogram body weight per day.

The desired dose is preferably presented as one, two, three or four or more subdoses administered at appropriate intervals throughout the day. These sub-doses may be administered in unit dosage forms, for example, containing about 0.5 to 2000 mg, preferably about 5, 25, 50, 150, 200, or 250 mg of active ingredient per unit dose form. While it is possible for the active ingredient to be administered alone, it is preferable to present it as a pharmaceutical composition. A further aspect of the present invention features pharmaceutical compositions comprising a compound of formula (1) or a pharmaceutically acceptable derivative thereof and a pharmaceutically acceptable carrier therefor.

The compositions of the present invention comprise at least one active ingredient, as defined above, together with one or more pharmaceutically acceptable carriers thereof and optionally other therapeutic agents. Each carrier must be “acceptable” in the sense of being compatible with the other ingredients of the composition and not deleterious to the recipient thereof Compositions include those suitable for oral, rectal, nasal, topical (including buccal and sublingual), vaginal or parenteral (including subcutaneous, intramuscular, intravenous and intradermal) administration.

The compositions may conveniently be presented in unit dosage form prepared by any of the methods well known in the art of pharmacy. Such methods include the step of bringing into association the active ingredient with the carrier which constitutes one or more accessory ingredients. In general, the compositions are prepared oy uniformly and intimately bringing in to association the active ingredient with liquid carriers or finely divided solid carriers or both, and then, if necessary, shaping the product.

Compositions of the present invention suitable for oral administration may be presented as discrete units such as capsules, cachets, sachets of granules or tablets (such as a swallowable, dispersible or chewable tablet) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Moulded tablets may be made by moulding in a suitable machine a mixture of the powdered compound moistened with an inert liquid diluent. The tablets may optionally be coated or scored any may be formulated so as to provide slow or controlled release of the active ingredient therein. Tablets may be enteric coated.

Compositions suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Compositions for rectal administration may be presented as a suppository with a suitable base comprising for example cocoa butter or a salicylate.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solution which may contain anti-oxidants, buffers, bacteriostats and solutes which render the formulation aqueous and non-aqueous sterile suspensions which may include suspending agents and thickening agents. The compositions may be presented in unit-dose or multidose sealed containers, for example, ampoules and vial, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

The active ingredient may also be presented in a composition comprising micrometer- or nanometer-size particles of active ingredient.

Preferred unit dosage compositions are those containing a daily dose or unit daily sub-dose (as herein above recited) or an appropriate fraction thereof, of the active ingredient. It should be understood that in addition to the ingredients particularly mentioned above the composition of this invention may include other agents conventional in the art having regard to the type of formulation in question, for example those suitable for oral administration may include flavoring agents or taste masking agents.

A further aspect of the invention relates to kits to be used in the treatment of patients suffering from viral infections. These kits include one or more oral dosage of a compound of formula (I) and may include one or more additional therapeutic agents. By way of illustration, a kit of the invention may include one or more tablets, capsules, caplets, gelcaps or liquid formulations containing a compound of formula (I) and one or more tablets, capsules, caplets, gelcaps or liquid formulations containing a compound of formula (I) in dosage amounts within the ranges described above. The kits may include as an insert printed dosing information for the co-administration of the agents.

The following examples are intended for illustration only and are not intended to limit the scope of the invention in any way.

### EXAMPLE 1

(1S,cis)-4-[2-Amino-6-(1-azetidinyl)-9H-purin-9-yl]-2-cyclopentene-1-methanol O-[phenyl(methoxy-L-alaninyl)]phosphoramidate

(a) (1S,cis)-4-[2-Amino-6-(1-azetidinyl)-9H-purin-9-yl]-2-cyclopentene-1-methanol

A solution of (1S,cis)-4-(6-chloro-9H-purin-9-yl)-2-cyclopentene-1-methanol (U.S. Pat. No. 5,917,041, June 1999) (300 mg, 1.13 mmol) and azetidine (1.00 g, 17.5 mmol) in methanol (10 mL) was maintained at 60° C. in a sealed Parr bomb for 18h. To the cooled solution was added 1 N sodium hydroxide (1.13 mL). Volatiles were evaporated and the residual solid chromatographed on silica gel. Elution with 6% methanol-chloroform gave title compound as a white solid, after trituration with acetonitrile (280 mg, 87%); m.p. 187-189° C.; [α]_{D}²⁰−31.9 (c 0.25, methanol); ¹H-NMR(DMSO-d₆) δ 7 59 (s, 1H), 6.11 (m, 1H), 5.86 (m, 3H), 5.37 (m, 1H), 4.75 (t, J=5 2 Hz, 1H), 4.24 (m, 4H), 3.44 (m, 2H), 2.85 (m, 1H), 2.60 (m, 1H), 2.36 (m, 2H), 1.55 (m, 1H).

Anal. Calcd. for C₁₄H₁₈N₆O: C, 58.73; H, 6.34; N, 29.35. Found: C, 58.74; H, 6.35; N, 29.43.

(b) (1S,cis)-4-[2-Amino-6-(1-azetidinyl)-9H-purin-9-yl]-2-cyclopentene-1-methanol O-[phenyl(methoxy-L-alaninyl)]phosphoramidate

Nucleosides were dried by evaporation of portions of dry pyridine. To a stirred solution of dry (1S,cis)-4-[2-amino-6-(1-azetidinyl)-9H-purin-9-yl]-2-cyclopentene-1-methanol (600 mg, 2.1 mmol) in dry pyridine (5 mL)-tetrahydrofuran (5 mL) under nitrogen was added 1 M tert-butylmagnesium chloride (3.2 mL). After 20 minutes, a solution of phenyl(methoxy-L-alaninyl)phosphorochloridate (prepared as described by C. McGuigan et al. J. Med. Chem. 1993, 36:1048-1052) (4.2 mL, 4.2 mmol) in anhydrous tetrahydrofuran was added and the solution was stirred at ambient temperature for 12 h. The resulting mixture was concentrated to a syrup under reduced pressure. This syrup was dissolved in dichloromethane (60 mL) and the organic phase washed with water (2×30 mL), then brine (30 mL) and dried (magnesium sulfate), filtered, and concentrated to a foam. This foam was purified by flash column chromatography on silica gel. The title compound was eluted with 5% methanol in chloroform to give, after removal of volatiles, 850 mg (77%) of a white solid foam; ¹H-NMR(DMSO-d₆) δ 7.58 (s, 1H), 7.34 (m, 2H), 7.16 (m, 3H), 6.05 (m, 5H), 5.4 (m, 1H), 4.24 (m, 4H), 3.95-4.10 (m, 2H), 3.85 (m, 1H), 3.57 (s, 3H), 3.07 (m, 1H), 2.57 (m, 1H), 2.36 (m, 2H), 1.6 (m, 1H), 1.2 (m, 3H); ³¹P-NMR(DMSO-d₆) 3.786, 3.439.

Anal. Calcd. for C₂₄H₃₀N₇O₅P.0.89H₂O: C, 53.03; H, 5.89; N, 18.04; Found: C, 53.03; H, 5.80; N, 17.94.

### EXAMPLE 2

(1S,cis)-4-[2-Amino-6-(cyclopropylmethylamino)-9H-purin-9-yl]-2-cyclopentene-1-methanol O-[phenyl(methoxy-L-alaninyl)]phosphoramidate

(a) (1S,cis)-4-[2-Amino-6-(cyclopropylmethylamino)-9H-purin-9-yl]-2-cyclopentene-1-methanol

A solution of (1S,cis)-4-(6-chloro-9H-purin-9-yl)-2-cyclopentene-1-methanol (U.S. Pat. No. 5,917,041, June 1999) (4.00 g, 15 mmol) and N-cyclopropyl-N-methylamine (J. Med. Chem., 1994, 37:3482-3491) (10.7 g, 37.7 mmol) in ethanol (60 mL) was refluxed for 2 h. To the cooled solution was added 1 N sodium hydroxide (15 mL). Volatiles were evaporated and the residual solid chromatographed on silica gel. Elution with 5% methanol-chloroform gave title compound as a white solid foam, after concentration of an ethanol solution (4.30 g, 95%); [α]_{D}²⁰−49.2 (c 0.23, methanol); ¹H-NMR(DMSO-d₆) δ 7.63 (s, 1H), 6.11 (m, 1H), 5.86 (m, 1H), 5.81 (br s, 2H), 5.41 (m, 1H), 4.74(t, J=5.2 Hz, 1H), 3.46 (m, 2H), 3.22 (m, 4H), 2.85 (m, 1H), 2.60 (m, 1H), 1.55 (m, 1H), 0.78 (m, 2H), 0.65 (m, 2H).

Anal. Calcd. for C₁₅H₂₀N₆O.0.25 C₂H₅OH.0.59H₂O: C, 57.73; H,: 7.09; N, 26.06. Found: C, 57.73; H, 6.99; N, 26.04.

(b) (1S,cis)-4-[2-Amino-6-(cyclopropylmethylamino)-9H-purin-9-yl]-2-cyclopentene-1-methanol O-[phenyl(methoxy-L-alaninyl)]phosphoramidate

The title compound was prepared by the method of Example 1, part b, from (1S,cis)-4-[2-amino-6-(cyclopropylmethylamino)-9H-purin-9-yl]-2-cyclopentene-1-methanol (part a of this example, 1.20 g, 3.70 mmol) and isolated as a white solid foam (1.30 g, 65%), after elution from a silica gel column with 5% methanol-chloroform; ¹H-NMR(DMSO-d₆) δ 7.61 (s, 1H), 7.34 (m, 2H), 7.16 (m, 3H), 5.8-6.1 (m 5H), 5.45 (m, 1H), 3.95-4.10 (m, 2H), 3.85 (m, 1H), 3.57 (s, 3H), 3.25 (m, 4H), 3.09 (m, 1H), 2.57 (m, 1H), 1.6 (m, 1H), 1.2 (m, 3H), 0.83 (m, 2H), 0.67 (m, 2H); ³¹P-NMR(DMSO-d₆) 3.766, 3.424.

Anal. Calcd. for C₂₅H₃₂N₇O₅P.0.14 CH₃CN/0.09H₂O: C, 55.32; H, 5.99; N, 18.22. Found: C, 55.31; H, 6.01; N, 18.22.

### EXAMPLE 3

(1S,cis)-4-(2-Amino-6-butoxy-9H-purin-9-yl)-2-cyclopentene-1-methanol O-[phenyl(methoxy-L-alaninyl)]phosphoramidate

(a) (1S,cis)-4-(2-Amino-6-butoxy-9H-purin-9-yl)-2-cyclopentene-1-methanol

To a solution of (1S,cis)-4-(6-chloro-9H-purin-9-yl)-2-cyclopentene-1-methanol (U.S. Pat. No. 5,917,041, June 1999) (4.00 g, 15 mmol) in dry n-butanol (50 mL) was added sodium hydride (60% in mineral oil, 723 mg, 18 mmol). The resulting mixture was maintained at 55° C. for 3 h. Volatiles were evaporated and the residual solid chromatographed on silica gel. Elution with 5% methanol-chloroform gave title compound as a white powder, after solidification from methanol-acetonitrile (3.70 g, 810%); [α]_{D}²⁰−77.5 (c 0.24, methanol); ¹H-NMR(DMSO-d₆) δ 7.75 (s, 1H), 6.36 (s, 2H), 6.11 (m, 1H), 5.87 (m, 1H), 5.42 (m, 1H), 4.73 (t, J=5.2 Hz, 1H), 4.40 (t, J=6.7 Hz, 2H), 3.44 (m, 2H), 2.86 (m, 1H), 2.60 (m, 1H), 1.73 (m, 2H), 1.60 (m, 1H), 1.41 (m, 2H), 0.94 (t, J=7.3 Hz, 3H).

Anal. Calcd. for C₁₅H₂₁N₅O₂.0.05CH₃CN.0.47H₂O: C, 57.78; H, 7.09; N, 22.54. Found: C, 57.77; H, 6.99; N, 22.53.

(b) (1S,cis)-4-(2-Amino-6-butoxy-9H-purin-9-yl)-2-cyclopentene-1-methanol O-[phenyl(methoxy-L-alaninyl)]phosphoramidate

The title compound was prepared by the method of Example 1, part b, from (1S,cis)-4-(2-amino-6-butoxy-9H-purin-9-yl)-2-cyclopentene-1-methanol (part a of this example, 1.00 g, 3.30 mmol) and isolated as a white solid foam (1.30 g, 75%), after elution from a silica gel column with 5% methanol-chloroform; ¹H-NMR(DMSO-d₆) δ 7.73 and 7.72 (both s, 1H), 7.34 (m, 2H), 7.16 (m, 3H), 6.38 (s, 2H), 6.05 (m, 3H), 5.46 (m, 1H), 4.40 (t, J=6.5 Hz, 2H), 3.95-4.15 (m, 2H), 3.85 (m, 1H), 3.57 (s, 3H), 3.07 (m, 1H), 2.65 (m, 1H), 1.7 (m, 3H), 1.4 (m, 2H), 1.2 (m, 3H), 0.93 (t, J=7.4 Hz, 3H); ³¹P-NMR(DMSO-d₆): 3.801, 3.459.

Anal. Calcd. for C₂₅H₃₃N₆O₆P.0.06CH₃CN.0.02H₂O: C, 55.12; H, 6.12; N, 15.51. Found: C, 55.12; H, 6.12; N, 15.51.

### EXAMPLE 4

(1S,cis)-4-(2-Amino-6-methoxy-9H-purin-9-yl)-2-cyclopentene-1-methanol O-[phenyl(methoxy-L-alaninyl)]phosphoramidate

(a) (1S,cis)-4-(2-Amino-6-methoxy-9H-purin-9-yl)-2-cyclopentene-1-methanol

To a solution of (1S,cis)-4-(6-chloro-9H-purin-9-yl)-2-cyclopentene-1-methanol (U.S. Pat. No. 5,917,041, June 1999) (4.00 g, 15 mmol) in dry methanol (60 mL) was added sodium hydride (60% in mineral oil, 1.2 g, 30 mmol). The resulting mixture was refluxed for 2 h. The solution was cooled (ice bath) while 1 N hydrochloric acid (15 mL) was added. Volatiles were evaporated and the residual solid chromatographed on silica gel. Elution with 5% methanol-chloroform gave title compound as a white solid foam, after evaporation of an ethanol solution (3.80 g, 97%); [X]D 20-102.5 (c 0.57, methanol); ¹H-NMR(DMSO-d6) δ 7.77 (s, 1H), 6.42 (s, 2H), 6.11 (m, 1H), 5.88 (m, 1H), 5.43 (m, 1H), 4.74 (t, J=5.2 Hz, 1H), 4.00 (s, 3H), 3.44 (m, 2H), 2.86 (m, 1H), 2.60 (m, 1H), 1.60 (m, 1H).

Anal. Calcd. for C₁₂H₁₅OH.0.21H₂O: C, 54.27; H, 6.21; N, 25.15. Found: C, 54.25; H, 6.26; N, 25.16.

(b) (1S,cis)-4-(2-Amino-6-methoxy-9H-purin-9-yl)-2-cyclopentene-1-methanol O-[phenyl(methoxy-L-alaninyl)]phosphoramidate

The title compound was prepared by the method of Example 1, part b, from (1S,cis)-4-(2-amino-6-methoxy-9H-purin-9-yl])-2-cyclopentene-1-methanol (part a of this example, 1.00 g, 3.80 mmol) and isolated as a white solid foam (1.30 g, 68%), after elution from a silica gel column with 3% methanol-chloroform; ¹H-NMR(DMSO-d₆) δ 7.74 and 7.73 (both s, 1H), 7.34 (m, 2H), 7.16 (m, 3H), 6.40 (br s, 2H), 5.95-6.05 (m, 6H), 5.46 (m, 1H), 3.9-4.1 (m, 2H), 3.95 (s, 3H), 3.85 (m, 1H), 3.57 (s, 3H). 3.1 (m, 1H), 2.65 (m, 1H), 1.65 (m, 1H), 1.2 (m, 3H); ³¹P-NMR(DMSO-d₆) 3.786, 3.439.

Anal. Calcd. for C₂₂H₂₇N₆O₆P.0.21H₂O: C, 52.20; H, 5.46; N, 16.60. Found: C, 52.20; H, 5.50; N, 16.52.

### EXAMPLE 5

(1S,cis)-4-[2-Amino-6-(propylamino)-9H-purin-9-yl]-2-cyclopentene-1-methanol O-[phenyl(methoxy-L-alaninyl)]phosphoramidate

(a) (1S,cis)-4-[2-Amino-6-(propylamino)-9H-purin-9-yl]-2-cyclopentene-1-methanol

A solution of (1S,cis)-4-(6-chloro-9H-purin-9-yl)-2-cyclopentene-1-methanol (U.S. Pat. No. 5,917,041, June 1999) (1.00 g, 3.77 mmol) and propylamine (3.1 mL, 38 mmol) in ethanol (16 mL) was refluxed for 3 h. To the cooled solution was added 5 N sodium hydroxide (0.76 mL). Volatiles were evaporated and the residual solid chromatographed on silica gel. Elution with 5% methanol-chloroform gave title compound as a white powder, after solidification from methanol-acetonitrile (1.00 g, 92%), [α]_{D}²⁰−57.1 (c 0.30, methanol); m.p. 146-148° C.; ¹H-NMR (DMSO-d₆) δ 7.59 (s, 1H), 7.16 (br s, 2H), 6.10 (m, 1H), 5.86 (m, 1H), 5.78 (s, 2H), 5.38 (m, 1H), 4.73 (t. J=5.2 Hz, 1H), 3.45 (m, 4H), 2.86 (m, 1H), 2.60 (m, 1H), 1.57 (m, 4H), 0.87 (t, J=7.4 Hz, 3H).

Anal. Calcd. for C₁₄H₂₀N₆O: C, 58.31; H, 6.99; N, 29.15. Found: C, 58.21; H, 7.05; N, 29.16.

(b) (1S,cis)-4-[2-Amino-6-(propylamino)-9H-purin-9-yl]-2-cyclopentene-1-methanol O-[phenyl(methoxy-L-alaninyl)]phosphoramidate

The title compound was prepared by the method of Example 1, part b, from (1S,cis)-4-[2-amino-6-(propylamino)-9H-purin-9-yl-2-cyclopentene-1-methanol (part a of this example, 1.00 g, 3.50 mmol) and isolated as a white solid foam (1.20 g, 64%), after elution from a silica gel column with 50% methanol-chloroform; ¹H-NMR(DMSO-d₆) δ 7.57 (s, 1H), 7.34 (m, 2H), 7.16 (m, 4H), 6.05 (m, 3H), 5.79 (br s, 2H), 5.41 (m, 1H), 3.95-4.15 (m, 2H), 3.85 (m, 1H), 3.57 (s, 3H), 3.38 (m, 2H), 3.07(m, 1H), 2.65 (m, 1H,m), 1.6 (m, 3H), 1.2 (m, 3H), 0.87 (t, J=7.2 Hz, 3H); ³¹P-NMR(DMSO-d6) 3.781, 3.455.

Anal. Calcd. for C₂₄H₃₂N₇O₅P 0.58H₂O: C, 53.38; H, 6.19; N, 18.16. Found: C, 53.38; H, 6.04; N, 18.04.

### EXAMPLE 6

(1S,cis)-4-[2-Amino-6-(isopropylamino)-9H-purin-9-yl]-2-cyclopentene-1-methanol O-[phenyl(methoxy-L-alaninyl)]phosphoramidate

(a) (1S,cis)-4-[2-Amino-6-(isopropylamino)-9H-purin-9-yl]-2-cyclopentene-1-methanol

A solution of (1S,cis)-4-(6-chloro-9H-purin-9-yl)-2-cyclopentene-1-methanol (U.S. Pat. No. 5,917,041, June 1999) (1.00 g, 3.77 mmol) and isopropylamine (3.2 mL, 38 mmol) in ethanol (16 mL) was refluxed for 4 h. To the cooled solution was added 5 N sodium hydroxide (0.76 mL). Volatiles were evaporated and the residual solid chromatographed on silica gel. Elution with 5% methanol-chloroform gave title compound as a white powder, after solidification from ethyl acetate—hexanes (1.00 g, 85%); [α]_{D}²⁰−50.6 (c 0.37, methanol); m.p. 87-90° C.; ¹H-NMR(DMSO-d₆) δ 7.59 (s, 1H), 6.88 (m, 1H), 6.10 (m, 4H), 5.85 (m, 1H), 5.77 (br s, 2H), 5.38 (m, 1H), 4.75 (t, J=5.2 Hz, 1H), 4.43 (m, 1H), 3.44 (m, 2H), 2.85 (m, 1H), 2.60 (m, 1H), 1.57 (m, 3H), 1.17 (d, J=6.4 Hz, 6H).

Anal. Calcd. for C₁₄H₂₀N₆O 0.0.07 EtOAc 0.19H₂O: C, 57.57; H, 7.08; N, 28.21. Found: C, 57.62; H, 7.08; N, 28.22.

(b) (1S,cis)-4-[2-Amino-6-(isopropylamino)-9H-purin-9-yl]-2-cyclopentene-1-methanol O-[phenyl(methoxy-L-alaninyl)]phosphoramidate

The title compound was prepared by the method of Example 1, part b, from (1S,cis)-4-[2-amino-6-(isopropylamino)-9H-purin-9-yl]-2-cyclopentene-1-methanol (part a of this example, 1.00 g, 3.10 mmol) and isolated as a white solid foam (1.00 g, 61%), after elution from a silica gel column with 5% methanol-chloroform; ¹H-NMR(DMSO-d₆) δ 7.57 (s, 1H), 7.34 (m, 2H), 7.16 (m, 3H), 6.90 (m, 1H), 6.05 (m, 3H), 5.79 (br s, 2H), 5.41 (m, 1H), 4.40 (m, 1H), 3.95-4.10 (m, 2H), 3.85 (m, 1H), 3.57 (s, 3H), 3.07 (m, 1H), 2.57 (m, 1H), 1.6 (m, 1H), 1.2 (m, 9H); ³¹P-NMR(DMSO-d6) 3.771, 3.444.

Anal. Calcd. for C₂₄H₃₂N₇O₅P 0.38H₂O: C: 53.74; H, 6.16; N, 18.28. Found: C, 53.74; H, 6.17; N, 18.16.

### EXAMPLE 7

(1S,cis)-4-[2-Amino-6-(allylthio)-9H-purin-9-yl]-2-cyclopentene-1-methanol O-[phenyl(methoxy-L-alaninyl)]phosphoramidate

(a) (1S,cis)-4-(2-amino-1,6-dihydro-6-thioxo-9H-purin-9-yl)-2-cyclopentene-1-methanol

(1S,cis)-4-(6-Chloro-9H-purin-9-yl)-2-cyclopentene-1-methanol (U.S. Pat. No. 5,917,041, June 1999) (481 mg, 1.81 mmol) and thiourea (151 mg, 1.99 mmol) were refluxed in n-propanol (6 mL) for 1.5 h. The resulting mixture was cooled (ice bath) and the precipitate collected after neutralization 1 N sodium hydroxide. Recrystallization from ethanol-water gave title compound as off-white powder (300 mg, 63%); m.p. >250° C.; [α]_{D}²⁰−29.1 (c 0.24, methanol).

Anal. Calcd. for C₁₁H₁₃N₅OS: C, 50.18; H, 4.98; N, 26.60; S, 12.18. Found: C, 50.20; H, 5.00; N, 26.54; S, 12.22.

(b) (1S,cis)-4-[2-Amino-6-(allylthio)-9H-purin-9-yl]-2-cyclopentene-1-methanol

Allyl chloride (0.43 mL) was added to a solution of (1S,cis)-4-(2-amino-1,6-dihydro-6-thioxo-9H-purin-9-yl)-2-cyclopentene-1-methanol (part a of this example, 210 mg, 0.80 mmol) in dioxane (2 mL) and 1 N sodium hydroxide (0.80 mL). The solution was stirred for 2 h. Water (10 mL)was added and the crude product was extracted into chloroform (3×10 mL). The chloroform extracts were dried (MgSO₄), volatiles evaporated, and the residual gum chromatographed on silica gel. Title compound eluted with 5% methanol-chloroform as an off-white solid foam (212 mg, 88%), after evaporation of an ethyl acetate solution; [α]_{D}²⁰−53.5 (c 0.245, methanol).

Anal. Calcd. for C₁₄H₁₇N₅OS 0.15 EtOAc 0.25H₂O: C, 54.61; H, 5.87; N, 21.81; S, 9.99. Found: C, 54.52; H, 5.92; N, 21.89; S, 9.93.

(c) (1S,cis)-4-[2-Amino-6-(allylthio)-9H-purin-9-yl]-2-cyclopentene-1-methanol O-[phenyl(methoxy-L-alaninyl)]phosphoramidate

The title compound was prepared by the method of Example 1, part b, from (1S,cis)-4-[2-amino-6-(allylthio)-9H-purin-9-yl]-2-cyclopentene-1-methanol (part b of this example, 700 mg, 2.30 mmol) and isolated as a white solid (1.10 g, 88%), after elution from a silica gel column with 3% methanol-chloroform and evaporation of an ethanol solution; MS (ES⁺) 545 (M+1); ³¹P-NMR(DMSO-d₆) δ 3.816,3.464.

Anal. Calcd. for C₂₄H₂₉N₆O₅PS 0.38 EtOH 0.45H₂O: C: 52.16; H, 5.69; N, 14.74. Found: C, 52.16; H, 5.59; N, 14.74.

### EXAMPLE 8

(1S,cis)-4-(2,6-Diamino-9H-purin-9-yl)-2-cyclopentene-1-methanol O-[phenyl(methoxy-L-alaninyl)]phosphoramidate

(a) (1S,cis)-4-(2,6-Diamino-9H-purin-9-yl)-2-cyclopentene-1-methanol

(1S,cis)-4-(6-Chloro-9H-purin-9-yl)-2-cyclopentene-1-methanol (U.S. Pat. No. 5,917,041, June 1999) (274 mg, 1.00 mmol) and ammonia (I, 40 mL) were stirred in a sealed Parr bomb at 60° C. for 3 days. After dilution with −78° C. methanol (40 mL), the ammonia was allowed to evaporate and 1 N sodium hydroxide (1.00 mL) was added. Volatiles were evaporated and the residual solid chromatographed on silica gel. Elution with 15% methanol-chloroform gave the title compound as a white powder (208 mg, 85%), after slurrying in acetonitrile; m.p. 174-176° C.; [α]_{D}²⁰−75.7 (c 0.25, methanol).

Anal. Calcd. for C₁₁H₁₄N₆O: C, 53.65; H, 5.73; N, 34.13. Found: C, 53.54; H, 5.73; N, 34.20.

(b) (1S,cis)-4-(2,6-Diamino-9H-purin-9-yl)-2-cyclopentene-1-methanol O-[phenyl(methoxy-L-alaninyl)]phosphoramidate

The title compound was prepared by the method of Example 1, part b, from (1S,cis)-4-(2,6-diamino-9H-purin-9-yl)-2-cyclopentene-1-methanol (part a of this example, 1.00 g, 4.06 mmol) and isolated as a white solid foam (730 mg, 37%), after elution from a silica gel column with 7.5% methanol-chloroform; ¹H-NMR(DMSO-d₆) δ 7.59 and 7.58 (both s, 1H), 7.34 (m, 2H), 7.17 (m, 3H), 6.73 (br s, 2H), 5.9-6.1 (m, 3H), 5.83 (br s, 2H), 5.41 (m, 1H), 3.95-4.10 (m, 2H), 3.85 (m, 1H), 3.58 (s, 3H), 3.09 (m, 1H), 2.65 (m, 1H), 1.64 (m, 1H), 1.21 (m, 3H);³¹P-NMR(DMSO-d₆) 3.796, 3.464.

Anal. Calcd. for C₂₁H₂₆N₇O₅P 0.06 CH₃CN 0.44H₂O: C: 50.95; H, 5.48; N, 19.86. Found: C, 50.95; H, 5.41; N, 19.86.

### EXAMPLE 9

(1S,cis)-4-[2-Amino-6-(cyclopropylmethylamino)-9H-purin-9-yl]-2-cyclopentene-1-methanol O-[phenyl(methoxy-α,α-dimethylglycinyl)]phosphoramidate

By the method of part b of Example 1, (1S,cis)-4-[2-amino-6-(cyclopropylmethylamino)-9H-purin-9-yl]-2-cyclopentene-1-methanol (part a of Example 2, 200 mg, 0.67 mmol) was treated with phenyl(methoxy-α,α-dimethylglycinyl)phosphorochloridate (1.7 mL of a 1 M solution in tetrahydrofuran, 1.7 mmoles; prepared as described by C. McGuigan et al. Antiviral Research 1997, 35:195-204). Title compound was isolated as a pale yellow solid foam (290 mg, 78%), after elution from a silica gel column with 3% methanol-chloroform; ¹H-NMR(DMSO-d₆) δ 7.62, 7.61 (both s, 1H), 7.34 (m, 2H), 7.18 (m, 3H), 6.06 (m, 1H), 5.95 (m, 1 H), 5.83 (m, 3H), 5.44 (m, 1H), 4.06 (m, 2H), 3.55 (s, 3H), 3.25 (m, 4r,), 3.09 (m, 1H), 2.6 (m, 1H), 1.6 (m, 1H), 1.2 (m, 6H), 0.79 (m, 2H), 0.67 (m, 2H); ³¹P-NMR(DMSO-d₆) 2.423, 2.388.

Anal. Calcd. for C₂₆H₃₄N₇O₅P 0.02 CH₃CN 0.12H₂O: C, 56.00; H, 6.19; N, 17.60. Found: C, 56.00; H, 6.26; N, 17.60.

### EXAMPLE 10

(1S,cis)-[2-Amino-6-(cyclopropylmethylamino)-9H-purin-9-yl]-2-cyclopentene-1-methanol O-[phenyl(benzyloxy-L-alaninyl)]phosphoramidate

By the method of part b of Example 1, (1S,cis)-4-[2-amino-6-(cycloprorylmethylamino)-9H-purin-9-yl]-2-cyclopentene-1-methanol (part a of Example 2, 200 mg, 0.67 mmol) was treated with phenyl(benzyloxy-L-alaninyl)phosphorochloridate (1.7 mL of a 1 M solution in tetrahydrofuran, 1.7 mmoles; prepared as described by C. McGuigan et al. Antiviral Research 1997, 35:195-204). Title compound was isolated as a pale yellow solid foam (160 mg, 39%), after elution from a silica gel column with 3% methanol-chloroform; ¹H-NMR(DMSO-d₆) δ 7.60 (s, 1H), 7.33 (m, 7H), 7.18 (m, 3H), 5.8-6.1 (m, 5H), 5.44 (m, 1H), 5.08 (m, 2H), 3.8-4.05 (m, 3H), 3.24 (s and m, 4H), 3.01 (m, 1H), 2.62 (m, 1H), 1.55 (m, 1H), 1.42 (m, 3H), 0.79 (m, 2H), 0.67 (m, 2H); ³¹P-NMR(DMSO-d₆) 4.047, 3.610.

Anal. Calcd. for C₃₁H₃₆N₇O₅P 0.15 CH₃CN 0.47H₂O: C, 59.46; H, 5.96; N, 15.84. Found: C, 59.45; H, 5.95; N, 15.84.

### EXAMPLE 11

(1S,cis)-4-[2-Amino-6-(cyclopropylmethylamino)-9H-purin-9-yl]-2-cyclopentene-1-methanol O-[phenyi(methoxy-L-phenylalaninyl)]phosphoramidate

By the method of part b of Example 1, (1S,cis)-4-[2-amino-6-(cyclopropylmethylamino)-9H-purin-9-yl]-2-cyclopentene-1-methanol (part a of Example 2, 200 mg, 0.67 mmol) was treated with phenyl(methoxy-L-phenylalaninyl)phosphorochloridate (1.7 mL of a 1 M solution in tetrahydrofuran, 1.7 mmoles; prepared as described by C. McGuigan et al. Antiviral Research 1997, 35:195-204). Title compound was isolated as a pale yellow solid foam (170 mg, 41%), after elution from a silica gel column with 3% methanol-chloroform; ¹H-NMR(DMSO-d6) 67.58 and 7.56 (both s, 1H), 6.95-7.3 (m, 10H), 5.8-6.2 (m, 5H), 5.405 (m, 1H), 3.7-4.0 (m, 2H), 3.53 (s and m, 4H), 3.25 (s and m, 4H), 2.9 (m, 1H), 2.45-2.8 (m, 5H), 1.55 (m, 1H), 0.79 (m, 2H), 0.67 (m, 2H); ³¹P-NMR(DMSO-d₆) 4.047, 3.610.

Anal. Calcd. for C₃₁H₃₆N₇O₅P 0.25 CH₃CN 0.40H₂O: C, 59.57; H, 5.96; N, 15.99. Found: C, 59.57; H, 5.93; N, 15.99.

### EXAMPLE 12

Anti-HIV Activity

Compounds were tested for anti-HIV activity in MT₄ cells according to the method described by Averett, D. R., J. Virol. Methods, 23, 1989, 263-276. Activity of the compounds against HIV was in the range IC₅₀ 0.010 μM—2.5 μM.

### EXAMPLE 13

Anti-Hepatitis B Virus Activity

Compounds were tested for anti-hepatitis B Virus activity according to the method described by Jansen, R. et al., Antimicrobial Agents and Chemotherapy Vol. 37, No. 3, pp. 441-447, 1993. Anti-hepatitis B activity of the compounds was in the range IC₅₀ 0.020,M -4.0 μM.

### EXAMPLE 14

### Tablet Formulation

The following formulations A, B and C are prepared by wet granulation of the ingredients with a solution of povidone, followed by addition of magnesium stearate and compression.

mg/tablet

Formulation A

| | |
|---|---|
| Active Ingredient | 250 |
| Lactose B.P. | 210 |
| Povidone B.P. | 15 |
| Sodium Starch Glycollate | 20 |
| Magnesium Stearate | 5 |
| | 500 |

Formulation B

| | |
|---|---|
| Active Ingredient | 250 |
| Lactose B.P. | 150 |
| Avicel PH 101 | 60 |
| Povidone B.P. | 15 |
| Sodium Starch Glycollate | 20 |
| Magnesium Stearate | 5 |
| | 500 |

Formulation C

| | |
|---|---|
| Active Ingredient | 250 |
| Lactose B.P. | 200 |
| Starch | 50 |
| Povidone | 5 |
| Magnesium Stearate | 4 |
| | 359 |

The following formulations, D and E, are prepared by direct compression of the admixed ingredients. The lactose in formulation E is of the direct compression type (Dairy Crest-“Zeparox”).

mg/tablet

Formulation D

| | |
|---|---|
| Active Ingredient | 250 |
| Pregelatinized Starch NF15 | 150 |
| | 400 |

Formulation E

| | |
|---|---|
| Active Ingredient | 250 |
| Lactose B.P. | 150 |
| Avicel | 100 |
| | 500 |

Formulation F (Controlled Release Formulation)

The formulation is prepared by wet granulation of the ingredients with a solution of povidone followed by the addition of magnesium stearate and compression.

| | mg/tablet |
|---|---|
| Active Ingredient | 500 |
| Hydroxypropylmethylcellulose | 112 |
| (Methocel K4M Premium) | |
| Lactose B.P. | 53 |
| Povidone B.P. | 28 |
| Magnesium Stearate | 7 |
| | 700 |

Drug release takes place over a period of about 6-8 hours and is complete after 12 hours.

### EXAMPLE 15

### Capsule Formulations

Formulation A

A capsule formulation is prepared by admixing the ingredients of formulation D in Example 6 above and filling into a two-part hard gelatin capsule. Formulation B (infra) is prepared in a similar manner.

mg/capsule

Formulation B

| | |
|---|---|
| Active Ingredient | 250 |
| Lactose B.P | 143 |
| Sodium Starch Glycollate | 25 |
| Magnesium Stearate | 2 |
| | 420 |

Formulation C

| | |
|---|---|
| Active Ingredient | 250 |
| Macrogel 4000 B.P. | 350 |
| | 600 |

Capsules of formulation C are prepared by melting the Macrogel 4000 B.P., dispersing the active ingredient in the melt and filling the melt into a two-part hard gelatin capsule.

Formulation D

| | mg/capsule |
|---|---|
| Active Ingredient | 250 |
| Lecithin | 100 |
| Arachis Oil | 100 |
| | 450 |

Capsules of formulation D are prepared by dispersing the active ingredient in the lecithin and arachis oil and filling the dispersion into soft, elastic gelatin capsules.

Formulation E

| | mg/capsule |
|---|---|
| Active Ingredient | 150.0 |
| Vitamin E TPGS | 400.0 |
| Polyethylene Glycol 400 NF | 200.5 |
| Propylene Glycol USP | 39.5 |

Four (4) kilograms (kg) of Vitamin E TPGS (obtained from Eastman Chemical Co.) was heated at 50° C. until liquefied. To the liquified Vitamin E TPGS, 2.005 kg of polyethylene glycol 400 (PEG400) (low aldehyde, <10 ppm, obtained from Union Carbide or Dow Chemical Co.) heated to 50° C. was added and mixed until a homogeneous solution was formed. The resultant solution was heated to 65° C. 1.5 kg of active ingredient was dissolved in the liquefied solution of Vitamin E TPGS and PEG 400. 0.395 kg of propylene glycol at room temperature was added and mixed until a homogenous solution was formed. The solution was cooled to 28-35° C. The solution was then de-gassed. The mixture was preferably encapsulated at 28-35° C. at a fill weight equivalent to 150 mg of volatiles-free compound, into Size 12 oblong, white opaque soft gelatin capsules using a capsule filling machine. The capsule shells were dried to a constant fill moisture of 3-6% water and a shell hardness of 7-10 Newtons, and placed in a suitable container.

Formulation F (Controlled Release Capsule)

The following controlled release capsule formulation is prepared by extruding ingredients a, b, and c using an extruder, followed by spheronization of the extrudate and drying. The dried pellets are then coated with release-controlling membrane (d) and filled into a two-piece, hard gelatin capsule.

| | mg/capsule |
|---|---|
| (a) Active Ingredient | 250 |
| (b) Microcrystalline Cellulose | 125 |
| (c) Lactose B.P. | 125 |
| (d) Ethyl Cellulose | 13 |
| | 513 |

### EXAMPLE 16

### Injectable Formulation

Formulation A

| | mg |
|---|---|
| Active Ingredient | 200 |
| Hydrochloric Acid Solution 0.1M or | 4.0 to 7.0 |
| Sodium Hydroxide Solution 0.1M q.s. to pH | |
| Sterile water q.s. to | 10 ml |

The active ingredient is dissolved in most of the water (35°-40° C.) and the pH adjusted to between 4.0 and 7.0 with the hydrochloric acid or the sodium hydroxide as appropriate. The batch is then made up to volume with water and filtered through a sterile micropore filter into a sterile 10 ml amber glass vial (type 1) and sealed with sterile closures and overseals.

Formulation B

| | | |
|---|---|---|
| Active Ingredient | 125 | mg |
| Sterile, Pyrogen-free, pH 7 Phosphate | 25 | ml |
| Buffer, q.s. to | | |

### EXAMPLE 17

### Intramuscular Injection

| | | |
|---|---|---|
| Active Ingredient | 200 | mg |
| Benzyl Alcohol | 0.10 | g |
| Glycofurol 75 | 1.45 | g |
| Water for injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml amber glass vials (type 1).

### EXAMPLE 18

### Syrup

| | | |
|---|---|---|
| Active Ingredient | 250 | mg |
| Sorbitol Solution | 1.50 | g |
| Glycerol | 2.00 | g |
| Sodium Benzoate | 0.005 | g |
| Flavor, Peach 17.42.3169 | 0.0125 | ml |
| Purified Water q.s. to | 5.00 | ml |

The active ingredient is dissolved in a mixture of the glycerol and most of the purified water. An aqueous solution of the sodium benzoate is then added to the solution, followed by addition of the sorbital solution and finally the flavor. The volume is made up with purified water and mixed well.

### EXAMPLE 19

### Suppository

| | mg/capsule suppository |
|---|---|
| Active Ingredient | 250 |
| Hard Fat, B.P. (Witepsol H15-Dynamit Nobel) | 1770 |
| | 2020 |

One-fifth of the Witepsol Hi 5 is melted in a steam-jacketed pan at 45° C. maximum. The active ingredient is sifted through a 200 μm sieve and added to the molten base with mixing, using a Silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 μm stainless steel screen and, with continuous stirring, is allowed to cool to 45° C. At a temperature of 38° C. to 40° C., 2.02 g of the mixture is filled into suitable, 2 ml plastic molds. The suppositories are allowed to cool to room temperature.

### EXAMPLE 20

### Pessaries

| | mg/pessary |
|---|---|
| Active Ingredient | 250 |
| Anhydrate Dextrose | 380 |
| Potato Starch | 363 |
| Magnesium Stearate | 7 |
| | 1000 |

The above ingredients are mixed directly.

### EXAMPLE 21

### Acid Stability

Compounds were tested for their stability towards acid-mediated hydrolytic decomposition employing a test designed to simulate stomach conditions. Each compound was incubated at an initial concentration of 0.3 mg/mL in dilute hydrochloric acid at pH 1 for 24 hours at 25° C. HPLC was run immediately for t=0 and at intervals up to approximately 24 hours. The results for compounds of Example 2, part b, Example 9, and for comparative phosphoramidates of 2′,3′-dideoxy-adenosine (Compound 1093) and 2′,3′-didehydro-2′,3′-dideoxy-adenosine (Compound 1001), described in PCT/GB96/00580, are given in the table below.

| Compound | Time (hr) | Compound left (%) |
|---|---|---|
| 1093 | 0 | 100 |
| | 13 | 0 |
| 1001 | 0 | 0 |
| | 17 | 0 |
| Example 2b | 0 | 100 |
| | 13 | 83 |
| | 24 | 74 |
| Example 9 | 0 | 100 |
| | 13 | 96 |
| | 24 | 94 |

Compound 1001 disappeared immediately (<1 minute). Compound 1093 degraded after less than 3 hours. The majority of the compounds of Example 2b and 9 remained intact after 24 hours.

1. A compound of formula (I) 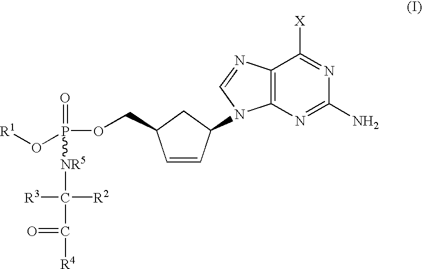
wherein:
R¹ is hydrogen; C₆₋₁₄aryl; or heteroaryl, optionally substituted with one or more substituents selected from the group consisting of C₁₋₆alkoxy, nitro, halogen, amino, hydroxy, carboxylate and esters thereof, carboxyalkyl, —CONHR⁶, and —CONR⁶R⁷, wherein R⁶ and R⁷, which may be the same or different, are independently selected from C₁₋₈alkyl, C₁₋₈alkylaryl or C₆₋₁₄aryl;
R² and R³ are independently selected from hydrogen or C₁₋₈alkyl, C₃₋₈cycloalkyl, C₂₋₈alkenyl, C₅₋₈cycloalkenyl, C₆₋₁₄aryl, or aralkyl wherein each C₁₋₈alkyl, C₃₋₈cycloalkyl, C₂₋₈alkenyl, C₅₋₈cycloalkenyl, C₆₋₁₄aryl or aralkyl may be optionally substituted with one or more substituents selected from the group consisting of C₁₋₈alkyl, halo, hydroxy, alkoxy, amino, aminoalkyl, aminodialkyl, —SH, thioalkyl, carboxylate and esters thereof, carboxyalkyl, —CONHR⁶, and —CONR⁶R⁷, wherein R⁶ and R⁷, which may be the same or different, are independently selected from C₁₋₈alkyl, C₁₋₈alkylaryl or C₆₋₁₄aryl; or R² and R³ can together form a 3- to 8-membered ring;
R⁴ is —OR⁸, —NR⁸R⁹ or —SR⁸, where R⁸ and R⁹, which may be the same or different, are independently selected from hydrogen, or C₁₋₈alkyl, C₃₋₈cycloalkyl, C₂₋₈alkenyl, C₅₋₈cycloalkenyl, or aralkyl, wherein each C₁₋₈alkyl, C₃₋₈cycloalkyl, C₂₋₈alkenyl, C₅₋₈cycloalkenyl, or aralkyl may be optionally substituted with one or more substituents selected from the group consisting of halo, hydroxy, alkoxy, amino, aminoalkyl, aminodialkyl, —SH, thioalkyl, carboxylate and esters thereof, carboxyalkyl, —CONHR⁶, and —CONR⁶R⁷, wherein R⁶ and R⁷, which may be the same or different, are independently selected from C₁₋₈alkyl, C₁₋₈alkylaryl or C₆₋₁₄aryl;
R⁵ is hydrogen; C₁₋₈alkyl; or C₆₋₁₄aryl; or R² and R⁵ may together form a 5- or 6-membered ring; or R³ and R⁵ may together form a 5- or 6-membered ring;
X is C₁₋₆alkoxy, optionally substituted by C₃₋₆cycloaklyl; C₃₋₈cycloalkyloxy; aryloxy, aralkyl or aralkyloxy in which the aryl may optionally be substituted with C₁₋₈alkyl, hydroxy, or halogen; C₃₋₆cycloalkylthio; C₁₋₈alkylthio; arylthio, or aralkylthio in which the aryl may optionally be substituted with C₁₋₈alkyl, hydroxy, or halogen; or X is a heterocyclic group containing an oxygen atom or one or two nitrogen atoms, and 3-7 carbon atoms with optional double bonds in the ring, optionally containing a sulfur and/or oxygen heteroatom and optionally substituted on the ring by one or more C₁₋₈alkyl, hydroxy or halogen groups, C₃₋₆cycloalkylthio, aralkylthio in which the aryl may be substituted with C₁₋₈alkyl, hydroxy or halogen; or X represents an imidazolylthio group in which the imidazolyl moiety may be substituted with C₁₋₈alkyl and/or C substituted with nitro; or X represents an amino group which is mono- or di-substituted by C₁₋₈alkyl, C₁₋₆alkoxy, hydroxyC₁₋₈alkyl and/or C₃₋₆cycloalkyl, C₆₋₁₄aryl, aralkyl, in which the C₆₋₁₄aryl may be optionally substituted with C₁₋₈alkyl, hydroxy, halogen or allyl optionally substituted with mono- or di-alkyl or alkoxy groups;
or a pharmaceutically acceptable derivative thereof; provided that X cannot be amino monosubstituted with cyclopropyl.

2. A compound of formula (I) according to claim 1 wherein R¹ is C₆₋₁₄aryl; R² and R³ are independently selected from hydrogen, C₁₋₈alkyl, aralkyl or C₆₋₁₄aryl optionally substituted with C₁₋₈alkyl; R⁴ is —OR⁸, where R⁸ is selected from C₁₋₈alkyl or aralkyl; R⁵ is hydrogen; and X represents C₁₋₆alkoxy or C₁₋₆alkylthio; or X represents a heterocyclic group containing a nitrogen atom, and 3-7 carbon atoms; or X represents an amino group which is mono- or di-substituted by C₁₋₆alkyl, C₁₋₆alkoxy, hydroxyC₁₋₆alkyl and/or C₃₋₆cycloalkyl, C₆₋₁₄aryl, aralkyl, in which the aryl may be optionally substituted with C₁₋₈alkyl, hydroxy, halogen or allyl optionally substituted with mono- or di-alkyl or alkoxy groups; or a pharmaceuticallly acceptable derivative thereof provided that X cannot be amino monosubstituted with cyclopropyl.

3. A compound selected from the group consisting of
(1S,cis)-4-[2-Amino-6-(1-azetidinyl)-9H-purin-9-yl]-2-cyclopentene-1-methanol O-[phenyl(methoxy-L-alaninyl)]phosphoramidate;
(1S,cis)-4-[2-Amino-6-(cyclopropylmethylamino)-9H-purin-9-yl]-2-cyclopentene-1-methanol O-[phenyl(methoxy-L-alaninyl)]phosphoramidate;
(1S,cis)-4-(2-Amino-6-butoxy-9H-purin-9-yl)-2-cyclopentene-1-methanol O-[phenyl(methoxy-L-alaninyl)]phosphoramidate;
(1S,cis)-4-(2-Amino-6-methoxy-9H-purin-9-yl)-2-cyclopentene-1-methanol O-[phenyl(methoxy-L-alaninyl)]phosphoramidate;
(1S,cis)-4-(2-Amino-6-(propylamino)-9H-purin-9-yl]-2-cyclopentene-1-methanol O-[phenyl(methoxy-L-alaninyl)]phosphoramidate;
(1S,cis)-4-[2-Amino-6-(isopropylamino)-9H-purin-9-yl]-2-cyclopentene-1-methanol O-[phenyl(methoxy-L-alaninyl)]phosphoramidate;
(1S,cis)-4-[2-Amino-6-(allylthio)-9H-purin-9-yl]-2-cyclopentene-1-methanol O-[phenyl(methoxy-L-alaninyl)]phosphoramidate;
(1S,cis)-4-(2,6-Diamino-9H-purin-9-yl)-2-cyclopentene-1-methanol O-[phenyl(methoxy-L-alaninyl)]phosphoramidate;
(1S,cis)-4-[2-Amino-6-(cyclopropylmethylamino)-9H-purin-9-yl]-2-cyclopentene-1-methanol O-[phenyl(methoxy-α,α-dimethylglycinyl)]phosphoramidate;
(1S,cis)-4-[2-Amino-6-(cyclopropyl methylamino)-9H-purin-9-yl]-2-cyclopentene-1-methanol O-[phenyl(benzyloxy-L-alaninyl)]phosphoramidate;
(1S,cis)-4-[2-Amino-6-(cyclopropyl methylamino)-9H-purin-9-yl]-2-cyclopentene-1-methanol O-[phenyl(methoxy-L-phenylalaninyl)]phosphoramidate;
and pharmaceutically acceptable derivatives thereof.

4. A compound according to any of claims 1 to 3 in the form of a single isomer.

5. A compound according to any of claims 1 to 3 in the form of a mixture of diastereomers.

6. A method of treating a virus infection in a human comprising administering to said human an effective anti-virus treatment amount of a compound of formula (I) according to any of claims 1 to 3 or a pharmaceutically acceptable derivative thereof.

7. The method according to claim 6 wherein the virus is selected from Human Immunodeficiency Virus, hepatitis B virus, and hepatitis C virus.

8. A method of treating a hepatitis B virus infection in a human comprising administering to said human an effective anti-hepatitis B treatment amount of a compound of formula (I) according to claim 1 or a pharmaceutically acceptable derivative thereof.

9. A method of treating a HIV infection in a human comprising administering to said human an effective anti-HIV treatment amount of a compound of formula (I) according to claim 1 or a pharmaceutically acceptable derivative thereof.

10. A compound according to any of claims 1 to 3 wherein the pharmaceutically acceptable derivative is a salt.

11. A pharmaceutical compostition comprising an effective anti-viral amount of a compound of formula (I) according to any of claims 1 to 3 or a pharmaceutically acceptable derivative thereof together with a pharmaceutically acceptable carrier therefor.

12. The pharmaceutical composition according to claim 11, further comprising an antiviral agent other than a compound of formula (I).

13. A pharmaceutical composition according to claim 11 or 12 in the form of a tablet or capsule.

14. A pharmaceutical composition according to claim 11 or 12 in the form of a solution, suspension or syrup.

15. A compound of formula (I) as claimed in claim 1 for use in medical therapy.

16. Use of a compound of formula (I) as claimed in claim 1 in the manufacture of a medicament for the treatment or prophylaxis of a virus infection.
